(19) United States
(12) Patent Application Publication
(10) Pub. No.: US 2020/0064318 A1
(43) Pub. Date: Feb. 27, 2020

(54) System, Method and computer program product for determining vehicle pollution and air quality index and communicating with other vehicles or transport or traffic management systems or equipment for Measuring, Monitoring and Controlling of Parameters to impact vehicle pollution and air quality index

(71) Applicant: Arya Deepak Keni, Woodbridge, NJ (US)

(72) Inventor: Arya Deepak Keni, Woodbridge, NJ (US)

(73) Assignee: Arya Deepak Keni, Woodbridge, NJ (US)

(21) Appl. No.: 16/112,650

(22) Filed: Aug. 25, 2018

(51) Int. Cl. G01N 33/00; G07C 5/00; G08G 1/01; G01C 21/34

(52) U.S. Cl. CPC G01N 33/0004; G01C 21/3469; G08G 1/0112; G07C 5/008

(57) ABSTRACT

This patent discloses methods, systems and computer program product for locally or remotely requesting, displaying and communicating real time vehicle pollution or air quality index determination of vehicles in operation, from a mobile device or non mobile device such as a smart phone or tablet; or non mobile device such as a computer; and communicating over a data network with other vehicles or transport systems or traffic management systems or devices for measuring, monitoring and controlling of vehicle pollution and air quality index parameters. The methods and systems use primary and secondary sources of data captured through user inputs or through sensors connected in operating vehicles or sensors capturing and communicating data connected to other vehicles or transport systems or traffic management systems in real time to calculate and communicate the vehicle pollution and air quality index parameters to end-user or other systems or devices or equipment for measuring, displaying, monitoring and controlling of parameters based on threshold limits and set parameters.

### RELATED APPLICATIONS

None

### BACKGROUND & FIELD OF THE INVENTION

### Field of the Invention

The System, Method and computer program product serves to utilize a network of software applications and hardware technologies with an aim to reduce air pollution by using technology of connected vehicles and connected transport systems and connected traffic management systems, more specifically by targeting one of the leading contributors of air pollution: vehicles and using emerging technologies such as interlinking hardware and software by wireless communication means to achieve this goal from a local or remote location. This set of systems and methods will provide the government entities, automotive manufacturers and related industries, and the vehicle operators and owners with valuable information and thus also an awareness (by quantifying once vaguely estimated data), to impose norms and use restrictive methods such as bollards and traffic management systems in an automated or non automated way, also to aid in achieving this goal of reducing vehicle pollution and improving air quality.

### Background of the Invention

Very few will disagree that pollution, especially that of the largest ‘sphere’ by volume, the atmosphere, is one of the biggest threats faced by humanity, mainly by its own doing, like vehicles releasing greenhouse gases, firecrackers, and so on. Our solution focuses on reducing harmful gaseous emissions from vehicles, through many futuristic, but quickly and effectively implementable methods.

To tackle the same problem of air pollution, people have designed various solutions, although what we encountered by research were mostly ways to directly abate it, such as catalytic converters which are enhanced organically, to render produced emissions harmless to health where usage of cheap catalysts and appropriate reactants render the gases such as carbon dioxide, or methane to other far less harmful products like carbon, or the production of a more sustainable Lithium ion battery, with a more conductive casing, for a longer battery time and eliminating any products whatsoever. Filters to ‘sieve’ air by Nano-particulate hexagonal netting is also under development, to directly make polluted air less polluted.

This set of systems and methods and computer program product aims to utilize technology used to monitor air pollution or traffic and further enhance them in advanced methods, to make it more accessible, informative, and effective in reducing pollution by emission and create user awareness first-hand on the matter.

### SUMMARY

### BRIEF SUMMARY OF THE INVENTION

The invention incorporates a number of known technologies into a novel system and method and computer program product for making vehicle pollution and air quality index determinations. More particularly, embodiments of the present invention use a mobile application client (an “App”) and ability for the mobile application client installed on a mobile device or non mobile device to perform search or calculations and communicate with operated vehicles or other vehicles or traffic management systems or transport systems over a data communications network. The proposed method, system and computer program product (including a web-computational tool (accessible with/out internet connection)) will address pollution problems by automating all possible ‘useful’ outputs by means of mathematical manipulation of functions and complex computations of numerous data inputs instantaneously chosen by the user (human being or system) or communicated by operated vehicle or or other vehicles or traffic management systems or transport systems.

The network of Systems and Methods and computer program product related to the category of connected vehicles and connected traffic management systems and connected transport systems comprises of the following major components and functionality:
1. To compute data such as the distance travelled, the optimum route to be travelled based on the path of least pollution, the amount of pollution in real time and through the whole path, and the contribution of that vehicle's emissions to the immediate environment, which will be on personal interfaces (smartphones, or vehicle tabs), or software in local area weather stations (Air Quality Index—AQI monitors and displays). This computation is on basis of real time data received from operated vehicles or other vehicles or traffic management systems or transport management systems captured through sensors
2. For a network of sensors using electromagnetic radiation (particularly low energy

Infrared), to aid in measuring, calculating, recording and communicating pollution related data, and pollutant profiles for each vehicle and of a collective area or region
3. For the restriction and optimization of vehicle travel in a particular area (city, an area of multiple zip-codes, and so on), including Bollards to restrict an excess of vehicles and toll stations alerted by the analyzed and collected data by above components 1&2, to hinder, discourage, and physically limit the vehicle density by automated or non automated way (human intervention)
4. For wireless interlinking of calculated, received, measured or monitored data through a secure network from all hardware and software that will do so or aid in doing so (like the sensors, bollards, AQI sensors and monitors and so on), to a centralized digital system to store large amounts of data, and be able to let users and systems access, help predict, and effectively format this data quickly and comprehensively for multiple uses to reduce vehicle pollution and improve air quality.
5. For descriptive and predictive data analytics regarding an enhancement to Google maps or any mapping application, to more accurately predict traffic conditions, with the additional notification of alerts for bollards, sensors, speed cameras and so on, along with a feature to calculate routes more economically, providing users and systems to choose path of travel with least vehicle pollution and improve air quality in areas along the path. This predictive analytical method can be used by the governing authority (government authorities such as road transport authorities and other related industries or organizations) to predict pollution conditions, set limits for vehicular density, determine safety limits by pollutants and area more accurately (due to much more data received to confidently model and impose)

All above technologies are able to communicate with each other over data communication network, and a method to abate pollution, while implementing emerging technologies to provide additional insights to the condition of the immediate atmosphere and techniques to reduce it (by predictive analytics) will also prove beneficial for future prevention and handling of air pollution regarding vehicles and traffic.

With the implementation of these technologies, vehicle operators, automotive industries, government organizations and governing authorities would be:
Better judge the condition of their vehicle in terms of emissions, and control the usage and repair of their vehicle in this regard
Having a more organized travel ‘pattern’ due to the mapping algorithm
Better evaluate norms or legislative restrictions based on the advanced method of gathering and compiling data conveniently and more accessibly
Better equipped to handle pollution-based situations for their vehicles, even while travelling
Target heavy polluting vehicles for repair or end of life disposal
More digitally reliable by the introduction of a cloud service (wireless data link and transfer technology) and a machine to machine communication network, thus aiding in user-friendliness and increased speed of data transfer and calculation
These major impacts on the lives of people will be made, when such a crucial technology is widely and aptly implemented.
   The computer program product will also contain additional tools and user guidance methods and systems which will be discussed in detail later (in the detailed description section).

These components are effectively accessible by the bundling algorithm, causing multiple such facets of specific code-language classes accessible under one domain. With button and click functionalities, recording inputs and switching between components, and communicating with other systems is made extremely simple.

This bundle-functionality domain is accessible with and without data network connection, in the form a web-computational tool online and in the form of an application (downloadable) offline; and send or receive data over a data network to other users, vehicles, systems or hardware or equipment

The method of data-processing in the bundle-functionality will be described in detail in the later sections. The method generally entails the way in which the computer program product will ‘flow’ the data through the program, giving the desired output.

These functionalities provided through the systems or computer program product can be easily linked via a data communication network to any other vehicles or relevant local or remote transport systems or traffic management systems or equipment, which will be discussed in detail in the following sections.

### BRIEF DESCRIPTION OF DRAWINGS

FIG. 1: Depicts the logic followed by the computer program product, and how the system and method for computational processes outputs pollution related data.

FIG. 2: Depicts how the software algorithm (on a mobile or non mobile computing device or a vehicular interface or transport system interface or traffic management system interface) interacts with relevant hardware for data inputs to provide outputs, essentially showing the process of gathering inputs.

FIG. 3: Shows the interlinking mechanism between many systems and methods to restrict and optimize vehicular traffic, and how data is conveyed between them.

FIG. 4: Shows the centralized system and method (described in claim 3) and how all the systems and methods depicted in the preceding figures are able to transfer, monitor, distribute, calculate, and provide data to a convenient technology for analysis and usage by the government or pollution authorities.

### SPECIFICATIONS

### DETAILED DESCRIPTION

This invention, incorporates many systems and methods of hardware and software and interlinks them by a system of hardware and software comprising of a wireless technology of interlinking and intercommunication between these systems and methods.

These sets of systems and methods will be discussed in detail referencing the figures in detail:

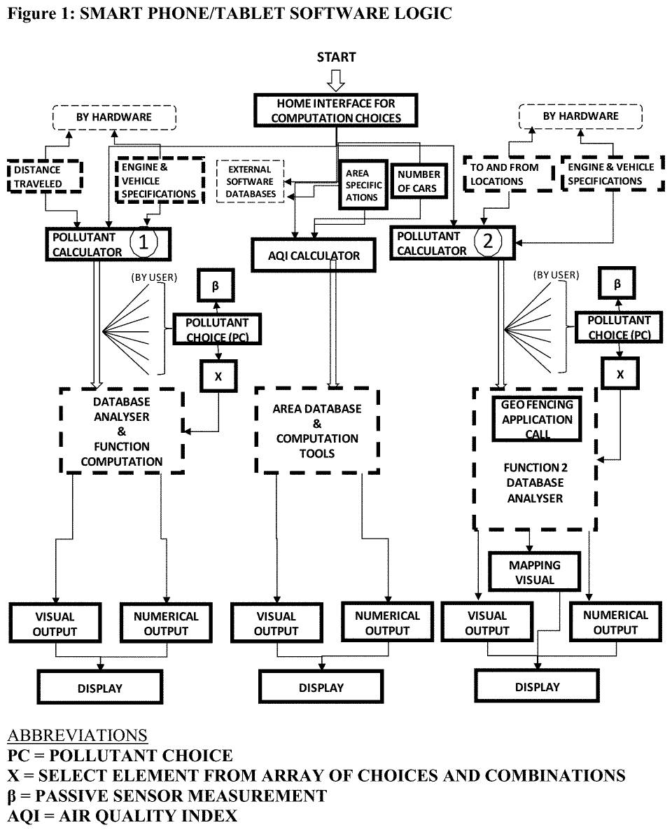

Referencing FIG. 1: The logic of data processing followed by the software computational tool is depicted, where external (hardware or software) inputs are received, and the algorithm decides between the 3 functionalities of AQI (Air Quality Index) calculation, and pollutant release by vehicle regarding 2 different methods. In the AQI calculation method, software data about the individual car, and about the area specifications (like number of cars in an area by GPS location monitoring, area occupied, number of lanes and road details like traffic signals and other vehicle associated road features) to calculate the factor of AQI the user's vehicle contributes to as well as a gauge of the safety level of the resultant AQI in real time. A numerical output as a whole number of the AQI resultant will be displayed along with the gauge with color coded ranges depicting the safety level, where the ranges are calculated by existing norms and statistical data, updated as and when the statistical data processing and area norms deem a different range for each safety level (safety level refers to cases such as: excellent, very good, good, OK, and so on). The 2 methods of calculating pollutant release by the vehicle are done as follows: the inputs from other hardware and software sources, such as carbon content in fuel data, vehicle model specifics like engine model and type, mileage statistics and so on, distance travelled by odometer, average and instantaneous velocity data storage, and air constituents from the RTO itself (online registry) to choose and calculate the amount of pollutant released (from a wide array of pollution causing gases and emittants, such as carbon monoxide, carbon dioxide, nitrogen monoxide and so on) along the distance travelled and gauge it on existing norms and statistical models specific to the distance travelled to determine how harmful the amount of released emission was to the immediate atmosphere (categorized by the area occupied by the specific zip codes traversed by the vehicle). This functionality is common for both methods of calculating pollutant release, where mathematical models and techniques (such as regression, and function computing) are used, but the difference lies in the method of input of distance travelled, and hence all travel related factors, such as traffic affecting speed for a particular time period, anticipated road length, and so on. The google maps library is utilized to accurately store and predict all of the above data by using the google satellite's geofencing algorithms and GUI (Graphical User Interface) visuals, and display to the user the map for a better sense of travel patterns. The most economical path can also be calculated by using the matrix of choices and possibilities of combination of pollutants chosen by the user or the software and use the fuel prices, mileage, carbon tax costs and so on to calculate the cost induced to the environment by the user chosen distance travelled (for that specific car model and properties), and thus using the data libraries (and mapping algorithms (shortest path finder, discrete route optimization and so on)) provided by google to find the least ‘harming’ route for the environment, also optimizing the amount of distance to be travelled. In the second method, only the distance travelled is taken as a raw input to calculate the cost and the amounts of each pollutants (chosen as an input) released, without the google maps functionalities. The methods are open for user choose, or when location sensing between ‘to’ and ‘from’ points is inaccurate (depending on connectivity issues or location specifics (hard to access place and so on)), but all the data is subliminally calculated for transfer via the wireless interlink to be finally accessed by the authorities. The user can choose what data can be presented on the screen, but all the data (for processing purposes to the government or pollution authorities) will be calculated pertaining to pollution and sent by storing it in the API chip on which this software runs. (Accessed software sources will also be sent for vehicle statistics to be utilized by the authorities). The sensors will be attached to the vehicle exhausts to measure and record the release of gas by one of the factors of gas expulsion (by exhausts; other methods including loss of gas from engine outlets and so on), and the measure data will be ready for transfer instantaneously (or as a sum over a period of time, to have a cumulative amount of data available at one time), due to the chips already existent in the sensors hardware and wiring. The sensors can be used to qualitatively and quantitively analyze pollutants, to rule out excess computations of non-existent pollutants specific to the vehicle. The measured volume by the sensors can be used in the software computational tool to augment the persisting calculations by providing additional units (density of gas released, percentage by volume contribution and so on since other parameters are calculated by other inputs), and be directly available as data ready to transfer.

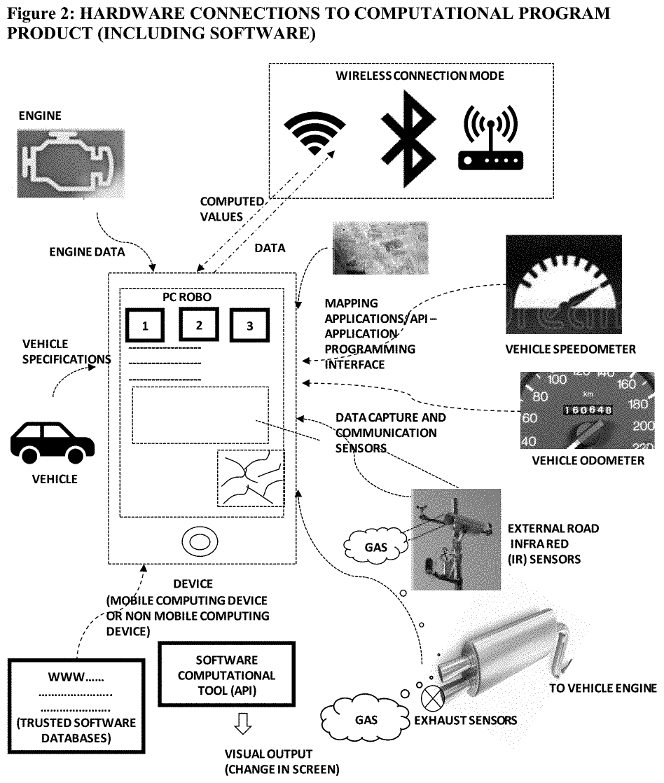

Referencing FIG. 2: The transfer of data via a wireless link of all the hardware and software sources to the computational software to serve as data inputs (to give an effective output) is shown here. All the software-based databases or detected values (like the sensors storing values in the CPU/RAM data chips (like the extremely popular and effective Arduino UNO chips) with a wireless transfer chip or the trusted online sources for vehicle and fuel data) can be directly accessed and transferred by the algorithm by coding relevant function and source ‘calls’ to the computational tool. All the other hardware tools recording data and storing them can have a wireless transfer chip with a CPU unit set (for effectively organizing data for a quicker data processing phase in the software computational tool in FIG. 1) for the transfer of data to the software algorithm. Any wireless networking agent (like wi-fi, Bluetooth, cellular network), can be utilized depending on the availability for the whole data transfer to the software computational tool. Listed and prominent hardware sources include: engines of the vehicles, speedometers, odometers and sensors (external or on exhausts), and software sources include car specifications, mapping software and online databases (trusted and approved).

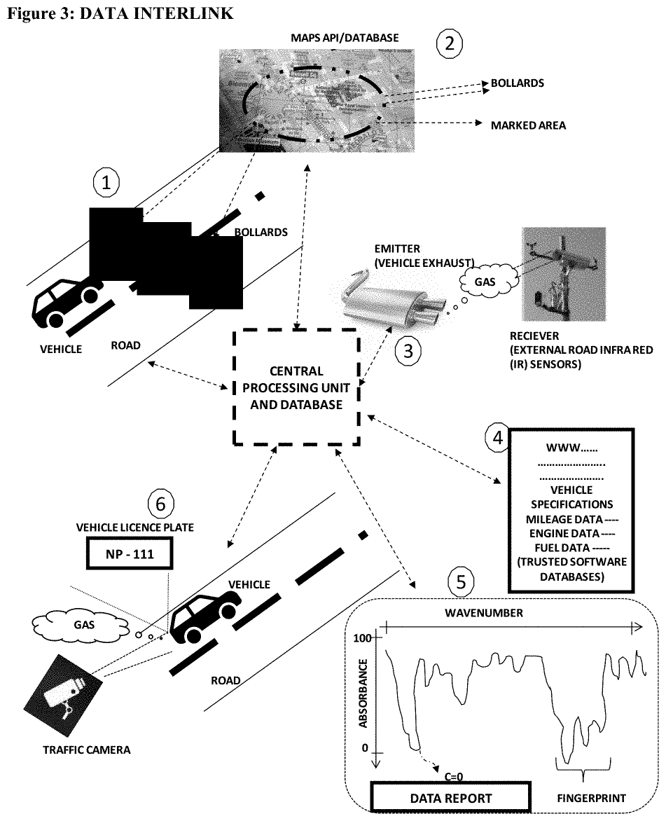

Referencing FIG. 3: The process of utilizing the processed data in figure depictions 1 and 2 to actually convey actions via a centralized data-flow unit (wireless and organized for correct data transfer) is extremely crucial. Data from external IR(infra red) sensors, which will analyze and profile atmospheric emissions using various chemical techniques such as IR spectroscopy and NMR spectroscopy (with a small data chip to analyze the received radiation after being absorbed by the emissions, and compare it to the existing experimental results for the compounds regarding the stated chemical techniques), direct stored data on online databases, traffic cameras capturing the credentials (like the number plate, color, and so on) of vehicles (paired up with the IR sensors to attach the analyzed emission data to a particular vehicle group based on the present vehicles at the time and the analyzed data at the time), and the mapping algorithm (which will be updated with use of all the newly processed data to show new features, such as: bollards and their activation state, traffic predictions based on pollution (and the environmentally safest route)). Preventive measures for traffic like bollards and pollution toll booths will be notified by the processed data about the safe limit of the vehicles in an area, the amount of pollution existing in an area, and so on, and will be programmed to activate if certain limits are exceeded (and vice-versa). Keep in mind that the bollards and the toll booths will have to be set-up by making amendments to existing roads, which may take some time, though the wireless transfer of data via the discussed chips (in FIG. 2) will significantly reduce the setup time by eliminating the need of wiring. Also, the placement of these features will have to be carefully sought by looking at the city map, to optimize the pathways in areas, and also such that any emergency situation can be handled (such as excess traffic having a certain emergency outlet, or emergency vehicles having special lane demarcations and so on). The large processing and computational power of the centralized processing unit and database will make all these actions instantaneously possible and make it easier for handling complex traffic situations (such as handling the state of various bollards on different roads).

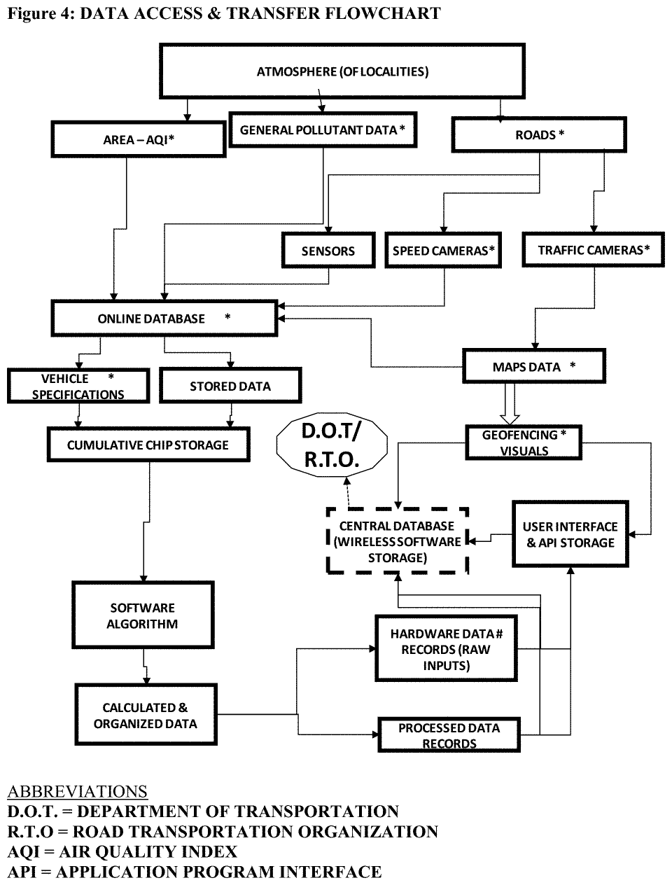

Referencing FIG. 4: The dealing of all of the above discussed data (in FIGS. 1,2 &3) may get really confusing even for some sophisticated software systems and methods. Thus, managing this vast amount of data, ranging from different numerical statistics, to hardware states, to vehicle specifics and so on needs a wireless software system which is easy to upload and transfer from, and easily accessible (like a sophisticated unlimited data storage plan for a ‘cloud’ service). All the stated data in FIG. 4 will flow from one place to the other, either to convey information (like for the bollards), or for processing data (like in the software algorithm), as depicted in FIG. 4. The figure comprehensively depicts how all the data from various modules will flow to the ‘cloud’ system, easily accessible to the authorities (government or RTO) (by a simple access key or password) for them to make more accurate norms, safety limits, and so on more easily (with all the data presented in a comprehensive fashion). The modules with ‘*’ symbol in FIG. 4 are pre-existing systems and methods (systems and methods that have been invented, but not utilized for the discussed reasons), which need to be slightly modified (mainly by providing the wireless capability data transfer chip), while the module with the ‘#’ symbol includes all the systems and methods like sensor data processes, bollard data processes, traffic and speed camera data processes, car exhaust data processes and so on (generally all the data coming from hardware systems and methods, and not software systems and methods).

This method is cost effective (IR sensors and data chips getting cheaper due to technological advancements), and most of its infrastructure (like sensors, speed cameras traffic cameras, static bollards and other such equipment) is virtually already available. Therefore, with all of these sets of systems and methods laid out in the described connections, then this invention (comprising of many different sub-systems and sub-methods) will provide useful data for the future and help tackle vehicular transportation systems, and ultimately, air pollution caused by it.

Thus, this system, method and computer program product offers a sophisticated and precise method of functioning, ultimately benefitting with huge amounts of data that is created and stored either locally or centrally for processing data on demand to analyze vehicle pollution patterns and air quality index patterns.

Thus this computer program product (or web-computational tool and application) offers accuracy and efficiency (in time and vastness of data processed) regarding vehicle pollution and air quality index data.

Regarding the hardware application, this computer program product is capable of a multi-device link to monitor, manage and control, providing effective additional uses of the computer program product to further benefit the environmental pollution control and vehicle technology development.

While specific ideas and embodiments have been illustrated and described, numerous modifications come to mind without significantly departing from the spirit of the invention, and the scope of protection is only limited by the scope of the accompanying claims.

### REFERENCES

1. Sensor Detection Technology, https://www.azosensors.com/article.aspx?ArticleID=339
2. Gas Detection, http://s7d9.scene7.com/is/content/minesafetyappliances/IR%20Gas%20Detection%20 Tech nology%20White%20Paper
3. Intelligent Transport Systems, http://civitas.eu/telematics/ITS
4. Air Quality Index, https://airnow.gov/index.cfm?action=aqibasics.aqi
5. Mapping Algorithms https://motherboard.vice.com/en_us/article/4x3pp9/the-simple-elegant-algorithm-that-makes-google-maps-possible
6. Smart Device Technology, https://www.computerworld.com/article/2593623/app-development/application-programming-interface.html

What is claimed is:

1. A system and method for monitoring, managing and controlling real time vehicle pollution and air quality index, the system comprising: a processor configured to execute a real time vehicle pollution and air quality index computer program product on a mobile or non mobile device; the real time vehicle pollution and air quality index computer program product configured to:
(1) Receive data from a user or another system that represents a plan to travel by vehicle, wherein the vehicle travel plan data comprises an origin and a destination,
(2) Receive a request to search or calculation for distance, vehicle pollution and air quality index determinations, the request comprising a keyword or multiple keywords or selections or values; based on receiving the request: retrieving, from storage circuitry, vehicle pollution and air quality index determinations search or calculation or calculator engine comprising of; a data apparatus or device comprising a plurality of database entries each corresponding to a respective vehicle pollution and air quality index determinations (such as non calculated or calculated values or data or metadata or digitized content), wherein each database entry comprises descriptive metadata associated with the respective vehicle pollution and air quality index calculations (such as non calculated or calculated values or data or metadata or digitized content); comparing, using control circuitry, the keyword or multiple keywords or selections or values to the descriptive metadata associated with each of the plurality of database entries; identifying, based on the comparing, a subset of the plurality of database entries that are associated with the descriptive metadata that includes the keyword or multiple keywords or selections or values, an application program interface to allow two way communication, interaction and data sharing between the computer program product and other relevant vehicle or transport or traffic management equipment or devices or systems,
(3) Determine a distance for the vehicle travel plan based at least in part on the received vehicle travel plan data,
(4) Determine an amount of pollutants released in real time (and as a cumulative amount over the distance travelled) by the vehicle based at least in part on the distance travelled from the point of origin, the vehicle speed, the type of vehicle, the engine composition of the vehicle, and the age and the condition of the vehicle
(5) Determine air quality index and provide that as input to vehicle travel plan data and determine number of vehicles and vehicle pollution in real time and provide that as input to vehicle travel plan data
(6) Retrieve vehicle travel mode data for a plurality of vehicle travel modes that satisfy the vehicle travel plan based at least in part on the received vehicle travel plan data, wherein the vehicle travel modes comprise a rental (or fleet taxi) vehicle travel mode and a personal vehicle travel mode,
(7) Compute a monetary cost for offsetting the vehicle pollution based on the vehicle travel plan data and the retrieved vehicle travel mode data, the computed monetary cost for offsetting the vehicle pollution being associated with a first vehicle travel mode option, the first vehicle travel mode option corresponding to travel by the rental vehicle from the origin to the destination in accordance with the vehicle travel plan,
(8) Compute a data value for another parameter for the vehicle based on the vehicle travel plan data and the retrieved vehicle travel mode data, the another parameter comprising a parameter other than monetary cost, the computed another parameter data value for the vehicle being associated with the first vehicle travel mode option,
(9) Compute a vehicle pollution and normalized cost for the first vehicle travel mode option as a weighted combination of the computed monetary cost and the computed another parameter data value for the rental (or fleet taxi) vehicle,
(10) Compute a monetary cost for offsetting the vehicle pollution of personal vehicle based on the vehicle travel plan data, the determined distance, and the retrieved vehicle travel mode data, the computed monetary cost for the personal vehicle being associated with a second vehicle travel mode option, the second vehicle travel mode option corresponding to travel by the personal vehicle from the origin to the destination in accordance with the vehicle travel plan and traffic patterns and air quality index of the target destination and calculated path towards target destination,
(11) Compute a data value for the another parameter for the personal vehicle based on the vehicle travel plan data and the retrieved vehicle travel mode data, the computed another parameter data value for the personal vehicle being associated with the second vehicle travel mode option,
(12) Compute a normalized cost for offsetting the vehicle pollution for the second vehicle travel mode option as a weighted combination of the computed monetary cost and the computed another parameter data value for the personal vehicle, and
(13) Make a selection as between the first vehicle travel mode option and the second vehicle travel mode option according to a vehicle travel mode selection rule based on the computed monetary cost for offsetting the vehicle pollution for the first and second vehicle travel mode options and traffic patterns and air quality index of the target destination and calculated path towards target destination.

2. The system and method according to claim 1 wherein: the vehicle pollution and air quality index determinations search or calculation or calculator computer program product or app is configured to read, receive and send data from said another sensor of said plurality of sensors; wherein the sensors connected to operated vehicle or other vehicles or cameras or infrared sensors, or sensors connected to other transport and traffic system devices; over a data communications network.

4. The system and method according to claim 1 wherein: the vehicle pollution and air quality index determinations search or calculation or calculator computer program product or app is configured to calculate vehicle pollution and air quality index determinations from received data from said another sensor of said plurality of sensors; wherein the sensors connected to operated vehicle or other vehicles or cameras or infrared sensors, or sensors connected to other transport or traffic system devices; over a data communications network.

5. The system and method according to claim 1 wherein: the vehicle pollution or air quality index determinations search or calculation or calculator app client further comprises wherein said at least one computer processor of the mobile device or non mobile device or non mobile device is configured to automatically communicate the vehicle pollution or air quality index determinations data to a pre-configured electronic address.

6. The system and method according to claim 1 wherein: the vehicle pollution or air quality index determinations search or calculation or calculator app client further comprises wherein said at least one computer processor of the mobile device or non mobile device or non mobile device is configured to allow the user or another equipment or device or system to send the vehicle pollution or air quality index determinations data to a user or another equipment or device or system-specified electronic address to monitor, manage or control the transport or traffic system device or devices or connected systems.

7. The system and method according to claim 1 wherein: the vehicle pollution or air quality index determinations search or calculation or calculator app client further comprises wherein said at least one computer processor of the mobile device or non mobile device or non mobile device is configured to display on the user or another vehicle connected device or system interface information or another transport or traffic system or devices or connected systems regarding the vehicle pollution or air quality index determinations data.

8. A system and method for managing and controlling traffic in real time in automated or non automated way based on vehicle pollution and air quality index comprising of:
(1) Vehicle Pollution and air quality index determination search or calculation or calculator computer program product or app on a mobile or non mobile device
(2) A processor or group of processors configured to execute a real time vehicle pollution and air quality index computer program product on a and mobile or non mobile device;
(3) Traffic cameras to record vehicle speed on the lane, and capture the license plate and credentials of the vehicle if violating the speed limit or any other road-traffic rules
(4) Infrared Sensors in Speed Cameras near lanes or on street lamps to detect and quantify the pollution data externally, and for a number of vehicles (as opposed to the system and method electronically calculating this data for an individual vehicle)
(5) Bollards or other restricting mechanisms or traffic management equipment or systems installed near junctions or around areas in a locality to restrict an overflow of vehicles into an area and optimize traffic and vehicle movement around the locality by using the data received and calculated by the systems and methods described in claim 1 and claim 8

9. The system and method according to claim 8 wherein: the vehicle pollution and air quality index determinations search or calculation or calculator computer program product or app is configured to read, receive and send data from said another sensor of said plurality of sensors; wherein the sensors connected to operated vehicle or other vehicles or cameras or infrared sensors, or sensors connected to other transport and traffic system devices; over a data communications network.

10. The system and method according to claim 8 wherein: the vehicle pollution and air quality index determinations search or calculation or calculator computer program product or app is configured to calculate vehicle pollution and air quality index determinations from received data from said another sensor of said plurality of sensors; wherein the sensors connected to operated vehicle or other vehicles or cameras or infrared sensors, or sensors connected to other transport or traffic system devices; over a data communications network.

11. The system and method according to claim 8 wherein: the vehicle pollution or air quality index determinations search or calculation or calculator app client further comprises wherein said at least one computer processor of the mobile device or non mobile device or non mobile device is configured to automatically communicate the vehicle pollution or air quality index determinations data to a pre-configured electronic address.

12. The system and method according to claim 8 wherein: the vehicle pollution or air quality index determinations search or calculation or calculator app client further comprises wherein said at least one computer processor of the mobile device or non mobile device or non mobile device is configured to allow the user or another equipment or device or system to send the vehicle pollution or air quality index determinations data to a user or another equipment or device or system-specified electronic address to monitor, manage or control the transport or traffic system device or devices or connected systems.

13. The system and method according to claim 8 wherein: the vehicle pollution or air quality index determinations search or calculation or calculator app client further comprises wherein said at least one computer processor of the mobile device or non mobile device or non mobile device is configured to display on the user or another vehicle connected device or system interface information or another transport or traffic system or devices or connected systems regarding the vehicle pollution or air quality index determinations data.

14. The system and method according to claim 8 wherein: the vehicle pollution or air quality index determinations search or calculation or calculator app client further communicates data to traffic management systems or traffic management personnel over mobile or non mobile device to operate the transport or traffic management systems to control movement of vehicles within affected areas in automated or non automated way.

15. A computer program product for managing, monitoring and controlling real time vehicle pollution and air quality index;
and for managing and controlling traffic;
embodied on a computer accessible medium configured to execute, on at least one computer processor of a mobile or non mobile device or non mobile device in communication or non communication with a vehicle pollution or air quality index determinations search or calculation platform over a communications network,
capable of remotely obtaining vehicle pollution or air quality index determinations data,
comprising: receiving, by the at least one computer processor, an interaction from a user or another equipment or device or system by communicating over data network;
calculating, by the at least one computer processor, vehicle pollution or air quality index determinations or values;
displaying to the user or another vehicle connected device or system interface information or another transport or traffic system or devices or connected systems regarding the vehicle pollution or air quality index determinations data in real time or when requested;
incorporating an application program interface to allow two way communication, interaction and data sharing between the computer program product and other relevant vehicle or transport or traffic management equipment or devices or systems,
wherein the at least one computer processor of the vehicle pollution or air quality index determinations search or calculation platform device is configured to receive the derived or calculated data from the vehicle pollution or air quality index determinations search or calculation or calculator app client via the data network interface, send the derived or calculated data to the vehicle pollution or air quality index determinations process parameters determination subsystem computer program product via the data network interface, receive the vehicle pollution or air quality index determinations data from the vehicle pollution or air quality index determinations process parameters determination subsystem via the data network interface, store the derived or calculated data and the vehicle pollution or air quality index determinations data in the vehicle pollution or air quality index determinations search or calculation database, and send the vehicle pollution or air quality index determinations data to the vehicle pollution or air quality index determinations search or calculation or calculator app client via the data network interface;
responding, by the at least one computer processor, to the user or another equipment or device or system interaction by reading sensor readings from another sensor of said plurality of sensors; wherein the sensors connected to other vehicle or transport or traffic management equipment or devices or systems, and another sensor, other than a touchscreen, a keyboard, and a mouse; forming, by the at least one computer processor, a vehicle pollution or air quality index determinations search or calculation request by inserting, by the at least one computer processor, the sensor readings from the plurality of sensors into the vehicle pollution or air quality index determinations search or calculation request; and sending, by the at least one computer processor, the vehicle pollution or air quality index determinations search or calculation request, over the communications network, to the vehicle pollution or air quality index determinations search or calculation platform;
and receiving, by the at least one computer processor, vehicle pollution or air quality index determinations data, from the communications network, in response to the vehicle pollution or air quality index determinations search or calculation request; and wherein the method comprises: retrieving, by the at least one computer processor, data comprising the at least one sensor reading; and calculating, by the at least one computer processor, vehicle pollution or air quality index determinations or values;
and for providing access to electronically stored vehicle pollution and air quality data and captured vehicle and transport and traffic systems data comprising of:
(1) A centralized online software to automatically or manually upload calculated and received data described in claim 1 and claim 2.
(2) Connection via any telecommunications service, such as Cellular networking, wi-fi, or Bluetooth, for the upload of data
(3) An Easy-Access method in the computer program product and software for quick data transfer and access for analysis and reception of the vehicle pollution and air quality data
(4) A system and method to interlink every related or connected device to calculate, monitor, receive, or store data via a wireless connection to aid in the quick data-transfer and data analysis process
While certain aspects of the disclosure are presented below in certain claim forms, the inventor contemplates the various aspects of the disclosure in any number of claim forms. For example, while only one aspect of the disclosure is recited as a means-plus-function claim under 35 U.S.C. .sctn.112, 6, other aspects may likewise be embodied as a means-plus-function claim, or in other forms, such as being embodied in a computer-readable medium. (Any claims intended to be treated under 35 U.S.C. .sctn.112, 6 will begin with the words “means for”.) Accordingly, the applicant reserves the right to add additional claims after filing the application to pursue such additional claim forms for other aspects of the disclosure.
